(19) United States
(12) Patent Application Publication
(10) Pub. No.: US 2022/0219008 A1
(43) Pub. Date: Jul. 14, 2022

(54) Photobiomodulation delivery devices for intracorporeal illumination via natural or surgically created orifices.

(71) Applicant: Nancy B. Lipko, Mayfield Heights, OH (US)

(72) Inventor: Nancy B. Lipko, Mayfield Heights, OH (US)

(73) Assignee: Nancy B. Lipko, Mayfield Heights, OH (US)

(21) Appl. No.: 17/174,485

(22) Filed: Feb. 12, 2021

Related U.S. Application Data
(60) Provisional application No. 63/135,601, filed on Jan. 9, 2021.

(51) Int. Cl. A61N 5/06

(52) U.S. Cl. CPC A61N 5/0603; A61N 2005/067; A61N 5/062

(57) ABSTRACT

A light emitting device is disclosed for intracorporeal Photobiomodulation (photostimulation, low-level light therapy, low-level laser therapy, phototherapy, photobiostimulation). The device will be removably inserted via a natural or surgically created orifice. The device will deliver therapeutic doses of electromagnetic radiation (EMR) light energy (photons) to cells, tissues, organs or body systems. The purpose of the device is to deliver light energy to cause a photochemical reaction for improving functionality, restoring functionality or causing a healing effect as needed for treating diseases, conditions or disorders of the body.

### SUMMARY OF THE INVENTION:

This patent is for a light emitting medical device intended for intracorporeal (inside the body) Photobiomodulation (photostimulation, low-level light therapy, low-level laser therapy, phototherapy, photobiostimulation). The device is intended to have an illuminated probe for removable insertion via a natural or surgically created orifice. The device will deliver therapeutic doses of electromagnetic radiation (EMR) light energy (photons) to cells, tissues, organs or body systems. The purpose of the device is to deliver light energy (to cause a photochemical reaction) for improving functionality, restoring functionality or causing a healing effect as needed for treating diseases, conditions or disorders of the body. The components of the device include the illuminated probe, a power and time control mechanism, (one or more wavelengths of) a light source, and a transducer to make use of power from a power supply (battery) or power outlet.

### BACKGROUND:

Photobiomodulation (PBM) is the intentional use of light as a treatment to living biological tissues. Light provides energy, in the form of photons, to elicit a chemical reaction in the living cell. As a result, the cell produces energy that can be used for cell differentiation, reproduction and growth. Photobiomodulation has also been called low-level light therapy (LLLT), low-level laser therapy (LLLT), and phototherapy (PT). PBM works for the body, roughly, like photosynthesis does for plants, algae and cyanobacteria.

In nature, photosynthesis, vision and the formation of vitamin D (using sunlight) are the most recognized occurrences of photochemistry. These photochemical reactions have been well defined through biological research.

Chloroplasts and mitochondria are both intracellular organelles. In eukaryotic cells, such as plants and algae, photosynthesis takes place in organelles called chloroplasts. In animals, mitochondria provide the host cell with the necessary molecular mechanisms to convert light energy (photons) to ATP for use by the host cell. Aerobic organisms evolved when mitochondria were able to provide the energy to sustain life, from within the cell, using oxidative phosphorylation, that did not require light.

Energy production in mitochondria (glycolysis) is known as oxidative phosphorylation, accomplished by oxidizing acetyl-CoA originating from carbohydrates using the citric acid cycle (Krebs cycle). This type of cellular respiration, known as aerobic respiration, is dependent on the presence of oxygen (O₂), water (H₂O) and coenzyme NAD+. All aerobic organisms use the citric acid cycle to produce ATP, thereby providing the principal source of energy for the cell. During the citric acid cycle NAD+ is reduced to NADH for use by the electron transport chain. Aerobic organisms have mitochondria in every type of tissues and cell. Typically, a multicellular eukaryote has hundreds or thousands of mitochondria in each of their cells. Human neurons are thought to contain about 2 million mitochondria per cell.

The most prominent roles of mitochondria are to produce the energy currency of the cell, ATP, through respiration, and to regulate cellular metabolism.⁸ Over time, photochemical reactions adapted to the cell's needs. Photosynthesis in plants and oxidative phosphorylation in animals occurred alongside other photochemical reactions necessary, such as those for vitamin D synthesis and sight. The various photochemical reactions that have evolved on the mitochondrial membrane can still be activated when light of the proper wavelength is made available to the mitochondria.

Adaptive mitochondrial functions in animals include:
Signaling through mitochondrial reactive oxygen species⁹
Regulation of the membrane potential⁸
Apoptosis-programmed cell death¹⁰
Calcium signaling (including calcium-evoked apoptosis)¹¹
Regulation of cellular metabolism¹²
Certain heme synthesis reactions¹³-such as those requiring porphyrin.
Steroid synthesis.
Hormonal signaling.¹⁵ Mitochondria are sensitive and responsive to hormones, in part by the action of mitochondrial estrogen receptors (mtERs). These receptors have been found in various tissues and cell types, including brain¹⁶ and heart¹⁷
Immune signaling¹⁸
Neuronal mitochondria also contribute to cellular quality control by reporting neuronal status towards microglia through specialized somatic-junctions¹⁹

In Photobiomodulation (PBM) the chemical substance that absorbs light is the respiratory enzyme cytochrome c oxidase, which is an integral part of the electron transport chain in mitochondria. Exposure to photons from several wavelengths of visible red light and near-infrared light have been demonstrated to improve both healing of skin wounds and remodeling of bones. For the past two decades there have been studies of red and near-infrared light for dental and periodontal procedures, orthodontics, and to prevent and treat oral mucositis (OM), a cancer treatment side effect. Studies are currently underway to understand the effects of different therapeutic wavelengths of light on various animal and human tissues including, but not limited to, the brain, the eye, muscles, tendons, skin, the immune system and the gastrointestinal tract. Mitochondrial dysfunction, mitochondrial structure, variations in mitochondrial volume, fluctuating mitochondrial distribution within a neuron, and other aspects of mitochondrial behavior are all currently under investigation.^{22,23,24,25,26}

Mechanisms of Photobiomodulation that have been proposed include the absorption of light by cytochrome c oxidase, a photochemical reaction that thereby causes a brief increase of reactive oxygen species, the release of nitrous oxide from cytochrome c oxidase and the opening of Ca⁺⁺ channels. These intracellular reactions are known to cause an increase in ATP production, an increase of the mitochondrial membrane potential, an increase of electron transport, the activation of signaling pathways, dilation of blood and lymph vessels, and the activation of transcription factors. The regulation of cytoprotective, antioxidant and anti-apoptotic pathways are all understood to be consequently affected. Inflammation and excitotoxicity are assumed to be downregulated. Steroid synthesis, immune signaling and hormonal signaling are subject to alteration.

### DETAILED DESCRIPTION:

This patent is for a light emitting medical device intended for intracorporeal (inside the body) Photobiomodulation (photostimulation, low-level light therapy, low-level laser therapy, phototherapy, photobiostimulation). The device is intended to have an illuminated probe for removable insertion via a natural or surgically created orifice. The device will deliver therapeutic doses of electromagnetic radiation (EMR) light energy (photons) to cells, tissues, organs or body systems. The purpose of the device is to deliver light energy (to cause a photochemical reaction) for improving functionality, restoring functionality or causing a healing effect as needed for treating diseases, conditions or disorders of the body. The components of the device include the illuminated probe, a power and time control mechanism, (one or more wavelengths of) a light source, and the ability use power from a power supply (battery) or power outlet.

The probe of the device (probe) is intended to be removably inserted via a natural or surgically created orifice for each treatment. The probe will provide light therapy to the targeted organ or tissue. The probe will be flexible, as needed. The probe will be designed to be atraumatic. The probe design is intended to be a light emitting cylinder composed of, covered by or encapsulated in biocompatible materials. The probe will deliver light in a radial fashion along (down the length of) a predetermined section of the probe. The treatment is expected to last, at the most, around 20 minutes from the time of probe insertion to the time of probe removal. The device dimensions will be adapted for various orifices, target tissues and organs. The device will be adapted for best practices regarding its methods of introduction or placement within the body.

The device will deliver light therapy to an area of interest (target). The device will deliver therapeutic doses of electromagnetic radiation (EMR) light energy (photons) to cells, tissues, organs or body systems. The purpose of the device is to deliver light energy to cause a photochemical reaction for improving functionality, restoring functionality or causing a healing effect as needed for treating diseases, conditions or disorders of the body. The light used will be rendered harmless for the user and the patient.

The probe will have the necessary rigidity (with or without a wire guide for placement), flexibility and shape for temporary placement inside the intended body cavity and/or organ via a natural or surgically created orifice such as the nose, mouth, external ear, anus, urethra, vagina, cervix, stoma, drain site, in a manner similar to a nasogastric tube, colonoscope, drain or a urinary catheter.

The probe will deliver light energy radially, providing a standardized light energy in a range of known therapeutic doses (J/cm²). One or more known therapeutic wavelengths of light will be used, selecting from the visible and near-infrared light spectra. Treatment dose will be adjustable, by time of exposure to light, to allow for optimization for a targeted tissue's treatment.

The probe will have an associated light source. The light source will be variously selected from an available range (in production) of light sources such as emitting diodes (LED) or lasers. Light delivered will be in the visible and/or near-infrared spectra. Illumination will consist of light of at least one wavelength in a therapeutic range from ultraviolet to near-infrared (189 nm to 1,300 nm). The probe will emit optical radiation in a range of therapeutic fluences (energy densities) between about 1 J/cm² and 10 J/cm². The light source will have an associated power control unit that is connected to a power source. There will be an electrical connection with the power source, such as a battery or energy transducer, for powering said light source.

The light photons delivered to the tissue make use of photochemical reactions that occur in the cells' mitochondria. The mitochondria exposed to light are temporarily activated to produce ATP via oxidative respiration. In response to photons of the selected wavelength(s) the mitochondrial citric acid cycle and associated electron transport chain, collectively known as oxidative respiration, are temporarily activated by the delivered photons to produce ATP for use as energy by the host cell. Additional cellular mechanisms may be triggered through byproducts and other photochemical reactions that involve reactive oxygen species, changes of membrane potentials, calcium signaling, heme synthesis reactions. Cellular functions including, but not limited to apoptosis (programmed cell death), metabolism, steroid synthesis, hormonal signaling and immune signaling may be altered temporarily for therapeutic purposes following treatment with light therapy.

The purpose of the device is to deliver therapeutic light doses for treatment of diseases, conditions, or disorders of the living body using natural and surgically created orifices. The device will be designed and manufactured to conform with practical standards for safety.

Orifices used for insertion of the probe may include, but are not limited to, the mouth, the nose, the outer ear, the urethra, the vagina, the cervix, the anus, stoma and/or drain sites. Target tissues and organs include, but are not limited to those of the 11 body systems:
1. Gastrointestinal tract
2. Respiratory tract
3. Urinary tract
4. Genital tract(s), both male and female
5. Endocrine system
6. Neurologic system
7. Muscular system
8. Lymphatic system
9. Integumentary system
10. Skeletal system
11. Cardiovascular system

Intended targeted tissues for Photobiomodulation include, but are not limited to, the esophagus, stomach, small intestine, large intestine, rectum, anus, lungs, bronchi, larynx, sinuses, bladder, prostate gland, uterus, vagina, spinal cord, brainstem, autonomic nervous system, pituitary gland, pineal gland, lymph nodes, and spleen. Intended targets may also include tissues in proximity to natural or surgically created orifices and targeted organs and structures, such as skin, muscle, connective tissue, peripheral nervous system, lymphatic vessels and blood vessels.

In addition, Photobiomodulation may be used to directly or indirectly cause changes in cell growth, cryoprotection, apoptosis, the inflammatory response, the immune system, immune signaling, cell and mitochondrial membrane potential, changes in excitotoxicity, Ca⁺⁺ signaling, regulation of cellular metabolism, heme synthesis reactions, steroid synthesis, hormonal signaling, blood and lymphatic vessel dilation, cellular quality control, and/or other signaling pathways.

1. A light emitting medical device intended for intracorporeal (inside the body) Photobiomodulation (photostimulation, low-level light therapy, low-level laser therapy, phototherapy, photobiostimulation, light therapy, photodynamic therapy) that is intended to have an illuminated probe for removable insertion via a natural or surgically created orifice in order to deliver therapeutic doses of electromagnetic radiation (EMR) light energy (photons) to cells, tissues, organs or body systems.

2. The device of claim 1 wherein the probe emits light energy in the form of photons for the purpose of causing a photochemical reaction that occurs in or around the cells of the targeted tissue for the purpose of modulation of one or more biological activities.

3. The device of claim 1 wherein the target of the light emitted can be the tissues in contact with or in close proximity (indirectly) to the body cavity or lumen being illuminated in addition to the cells, tissue, body organs or body systems being (directly) illuminated.

4. The device of claim 1 wherein the light emitted can be used for Photobiomodulation, low-level light therapy, low-level laser therapy, light therapy, phototherapy, photobiostimulation, laser radiation, or photo-dynamic therapy (PDT).

5. The device of claim 1 wherein the intention is to enhance the health of cells, tissues or organs in order to enable the body's ability to improve and/or restore functionality, heal, cure, slow progression or remedy diseases, conditions, or disorders of the living body.

6. The device of claim 1 consisting of a light emitting probe, a controller for power, a controller for time of illumination, a controller for wavelengths of light emitted, a light source and a power supply and a mechanism, such as a transducer, to adapt power from a power supply (battery) or power outlet.

7. The probe of claim 1 wherein the probe is intended to be removably inserted via a natural or surgically produced (such as stoma, drain site) orifice for the purpose of treatment.

8. The probe of claim 1 wherein the probe's external surface is biocompatible, flexible and/or rigid and is capable of adapting to the contour of the body passages targeted, whose placement is atraumatic.

9. The probe of claim 1 wherein the probe does and/or does not require a wire guide for placement and does and/or does not include a method for visualization of the surrounding tissues.

10. The probe of claim 1 wherein the light is emitted in a radial fashion along (down the length of) a predetermined section of the probe, is of at least one wavelength of visible or near-infrared light.

11. The probe of claim 1 wherein the probe is removed after selected time/dose(s) of light therapy is/are delivered.

12. The probe of claim 1 wherein the probe is configured to provide light therapy to at least one cavity of the patient's body, such cavity being at least one of a gastrointestinal, a urological, a respiratory, a genital, a cranial (such as the middle ear), a spinal, a pelvic, an abdominal, a thoracic via a natural or surgically created (stoma) orifice.

13. The probe of claim 1 wherein the design is for insertion via the nose, mouth, outer ear, urethra, vagina, cervix, anus, an existing stoma, a drain site, or an existing fistula (fistula including but not limited to anovaginal fistula: between the anal canal and vagina; colovaginal fistula: between the vagina and colon; colocutaneous fistula: between the colon and the epidermis).

14. The probe of claim 1 wherein the dimensions of the probe are predetermined by the intended use.

15. The probe of claim 1 wherein the probe can be inserted into an orifice in the manner of a nasogastric tube, urinary catheter, tympanostomy tube or drainage tube.

16. The probe of claim 1 wherein the light emitted is for a therapeutic purpose.

17. The probe of claim 1 wherein the light energy transmitted is from a light source embedded in the probe and/or the light energy is propagated from a designated light source to the probe, the light energy emitted is in the range of 0.1-20 J/cm², and the therapeutic range of light energy dose delivered is roughly the range of 1-10 J/cm²

18. The power source of claim 1 wherein the source is either an independent power source, such as a battery, or an energy transducer for use of an electrical outlet.

19. The device of claim 1 wherein the light source is one or more laser, light emitting diodes (LED), lamp, superluminous diodes or laser diode, or any combination of appropriate light sources available for wavelength(s) selected from the visible and near-infrared spectrum.

20. The device of claim 1 wherein said controls are used to determine light therapy parameters selected from a group comprising dose/time, wavelength(s) and power (on/off).

21. The device of claim 1 wherein the device is tested for and meets applicable safety standards and is harmless when used in its intended manner.
